(19) United States
(12) Patent Application Publication
(10) Pub. No.: US 2009/0074676 A1
(43) Pub. Date: Mar. 19, 2009

(54) Inhibition of p38 MAPK For Treatment Of Obesity

(75) Inventor: Baichun Yang, Durham, NC (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION

(21) Appl. No.: 11/915,008

(22) PCT Filed: May 22, 2006
(86) PCT No.: PCT/US2006/019866

§ 371 Date: Jun. 26, 2008

Related U.S. Application Data
(60) Provisional application No. 60/683,751, filed on May 23, 2005.

(51) Int. Cl. A61K 9/12; A61K 31/501; A61K 31/519; A61K 38/28; A61K 38/22

(52) U.S. Cl. 424/45; 514/256; 514/264.1; 514/4; 514/12

(57) ABSTRACT

The present invention is directed to the novel use of an inhibitor of CSBP/p38 Kinase for the treatment of obesity, and reducing weight loss in a mammal.

### FIELD OF THE INVENTION

The present invention relates to a novel use of inhibitors of the p38 kinase for the treatment of certain diseases and conditions.

### BACKGROUND OF THE INVENTION

The p38 MAPK (p38) family consists of two subgroups: p38α and β isoforms (sensitive to the class of pyridyl imidazole such as those represented by 4-(4-Fluorophenyl)-2-(4-methylsulfinylphenyl)-5-(4-pyridyl)imidazole and found in WO 93/14081, and WO95/02591) p38γ and δ (which are deemed to be generally insensitive to the class of pyridyl imidazoles as described above (See, Nebreda et al., Trends Biochem Sci 2000, 25: 257-260). All p38 isoforms share many molecular targets and possess a common TGY phosphorylation and activation motif (See Ono et al., Cell signal 2000, 12: 1-13). Their direct activators are mitogen-activated protein kinase kinase 3/6 (MKK3/6) (See, Enslen et al., J Biol Chem 1998, 273: 1741-1748). The p38 is characteristically induced by activation of cellular stress-mediated signaling pathways to modify inflammation, cell growth, and apoptosis (See Ono et al, Supra; Roux et al., Microbiol Mol Biol Rev 2004, 68: 320-344; Kumar et al., Nat Rev Drug Discov 2003, 2: 717-2; Irving et al., J Cereb Blood Flow Metab 2002, 22: 631-647; and Nakagami et al., Diabetes. 2001, 50:1472-1481).

The importance of major cellular signaling pathways in control of metabolism, particularly adipogenesis, gluconeogenesis and related pathophysiology has recently been investigated. p38 alpha has been implicated as one of the pathways involved in these metabolic pathways and thus may have utility in diseases related to metabolic disorders, such as obesity, diabetes, or metabolic syndrome. In addition, metabolic diseases are now understood to have an inflammatory compontent. A role of the p38 MAPK stress pathway is therefore also consistent with the pathophysiology of these diseases. (Fujishiro M, et al., J Biol Chem 2001, 276: 19800-198068-14; Huang C, et al., Diabetes 2002, 51: 2090-2098; Somwar et al., J Biol Chem. 2002, 277: 50386-50395; Carlson et al., Diabetes 2003, 52: 634-641; Gum et al., Mol Endocrinol. 2003,17:1131-1143; Cao et al., 64^{th} ADA Scientific Session, Later break, Orlando, Jun. 4-8, 2004; and Cao et al., Keystone Symposim, p41, #107, January, 2005.

Obesity is not only a nutritional disorder in Western societies, it is also a serious health concern because of its association with adult-onset diabetes, hypertension, and heart disease. Obesity is currently described by World Health Organization (WHO) as an epidemic in many 25 industrialized nations. While there is evidence to suggest that body weight was physiologically regulated, the molecular mechanism has remained elusive. Obesity, if left unabated, can have dire health consequences, such as adult-onset diabetes (Type II diabetes), hypertension, heart disease, osteoarthritis, increased blood pressure, increased incidence of stroke, and accelerated morbidity and mortality.

Consequently, innovative approaches are urgently needed at both the basic science and clinical levels to treat obesity. There remains a need in this area for compounds which are cytokine suppressive anti-inflammatory drugs, i.e. compounds which are capable of inhibiting the CSBP/p38/RK kinase, in the treatment of obesity and weight loss.

### DETAILED DESCRIPTION OF DRAWINGS

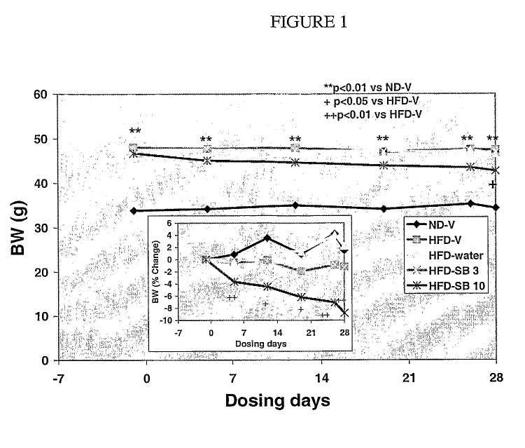

FIG. 1 demonstrates AKR mice on high fat diet for 14-15 weeks having a stable weight 40% greater than normal diet controls. Compound 2, trans-1-(4-hydroxycyclohexyl)-4-(4-fluorophenyl)-5-[(2-methoxy)pyrimidin-4-yl]imidazole decreased BW in the 18-19 weeks old DIO AKR mice. ND-V: Normal diet-Vehicle. HFD-V: High fat diet-Vehicle. HFD-water: High fat diet-water. HFD-SB 3: High fat diet and Compound 2, administered at 3 mg/kg. HFD-SB 10: High fat diet, and Compound 2, administered at 10 mg/kg.

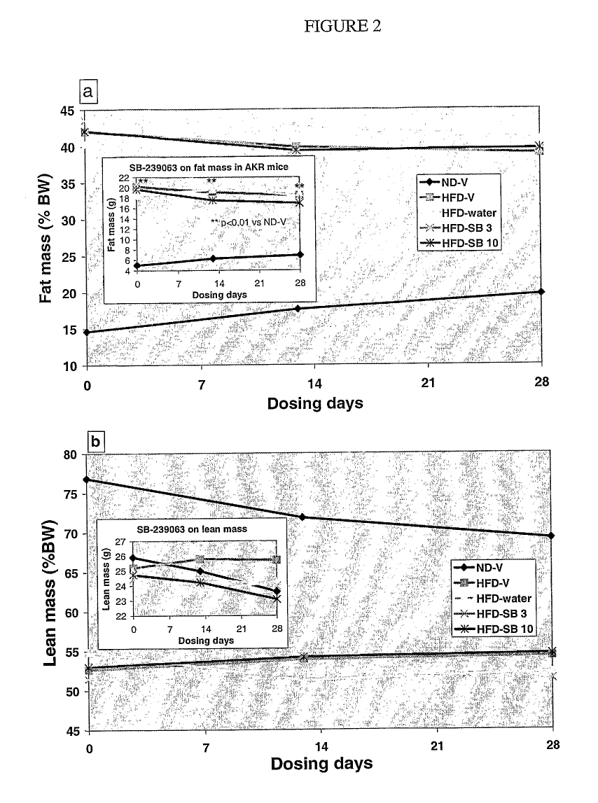

FIG. 2 demonstrates AKR mice on high fat diet for 14-15 weeks had a double fat mass and smaller lean mass comparing with normal chow controls. Compound 2, SB 239063 or trans-1-(4-hydroxycyclohexyl)-4-(4-fluorophenyl)-5-[(2-methoxy)pyrimidin-4-yl]imidazole tended to decrease fat mass (a) but did not affect lean mass (b) in the 18-19 weeks old DIO AKR mice. ND-V: Normal diet-Vehicle. HFD-V: High fat diet-Vehicle. HFD-water: High fat diet-water. HFD-SB 3: High fat diet-Compound 2, administered at 3 mg/kg. HFD-SB 10: High fat diet-Compound 2, administered at 10 mg/kg.

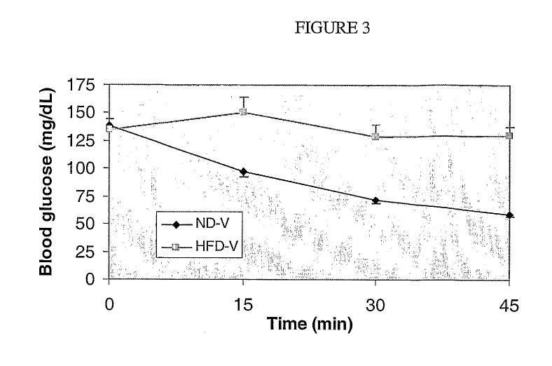

FIG. 3 demonstrates AKR mice on a high fat diet for 14-15 weeks showing decreased response during an insulin tolerance test. ND-V: Normal diet-Vehicle. HFD-V: High fat diet-Vehicle. Insulin: i.p., 0.75 U/kg.

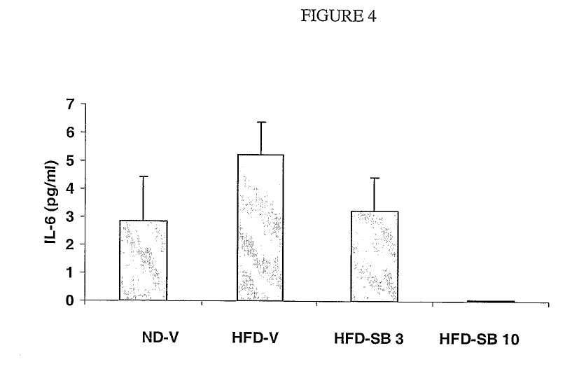

FIG. 4 demonstrates AKR mice on a high fat diet for 14-15 weeks having a higher serum IL-6 level than normal chow controls. Compound 2 showed decreased serum IL-6 in 18-19 weeks old DIO AKR mice. ND-V: Normal diet-Vehicle. HFD-V: High fat diet-Vehicle. HFD-SB 3: High fat diet-Compound 2 administered at 3 mg/kg. HFD-SB 10: High fat diet-Compound 2 administered at 10 mg/kg.

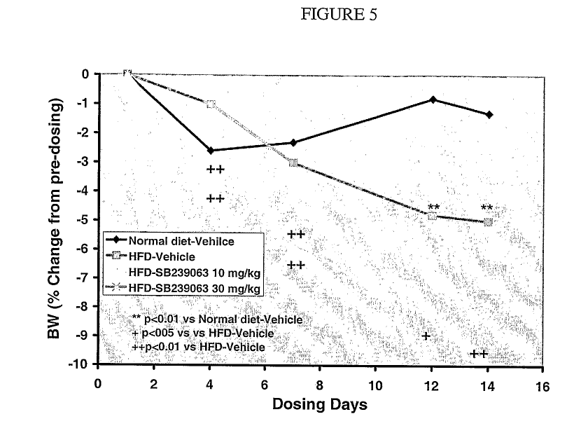

FIG. 5 demonstrates Compound 2 showing decreased BW in the 24-25 weeks old DIO AKR mice. HFD: High fat diet.

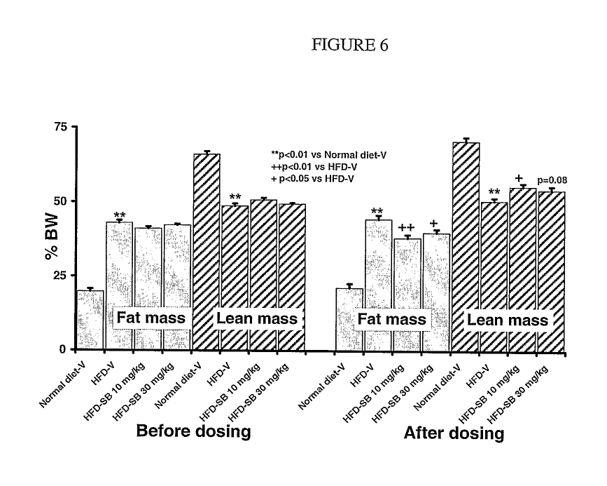

FIG. 6 AKR mice on high fat diet for 18-20 weeks had a double fat mass as normal diet controls. Compound 2 showed decreased fat mass in 24-25 weeks old DIO AKR mice. HFD: High fat diet. HFD-V: High fat diet-Vehicle. HFD-SB: High fat diet-Compound 2.

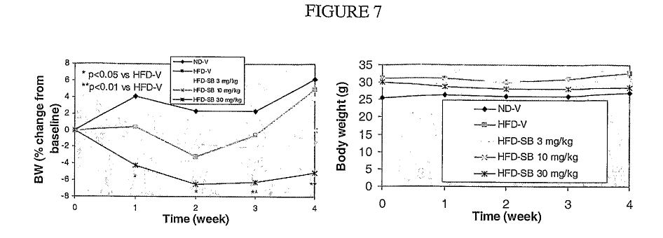

FIG. 7 demonstrates C57BL/6 mice on a high fat diet for 11-15 weeks having a stable weight ˜20% greater than normal diet controls. Compound 2 showed decreased body weight in DIO C57BL/6 mice. ND-V: Normal diet-Vehicle. HFD-V: High fat diet-Vehicle. HFD-SB: High fat diet-Compound 2.

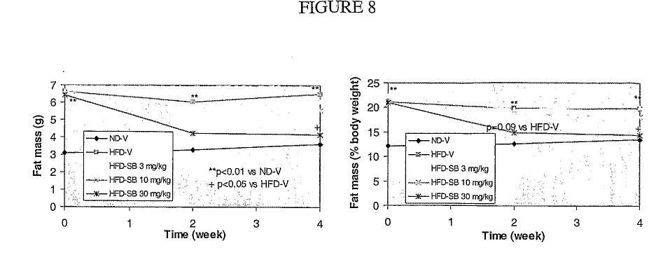

FIG. 8 demonstrates C57BL/6 mice on a high fat diet for 11-15 weeks having a double fat mass as normal diet controls. Compound 2 showed decreased body weight in DIO C57BL/6 mice. ND-V: Normal diet-Vehicle. HFD-V: High fat diet-Vehicle. HFD-SB: High fat diet-Compound 2.

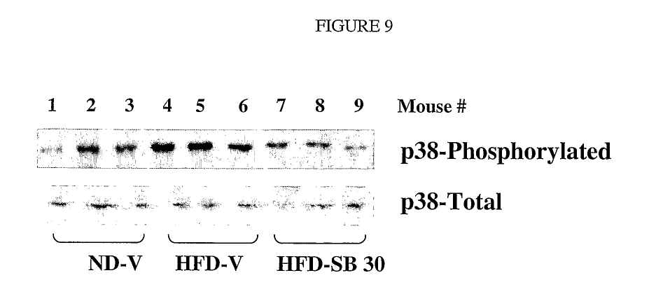

FIG. 9 demonstrates a Western analysis showing a marked increase in p38 phosphorylation in the liver of C57BL/6 mice on high fat diet for 14-15 weeks comparing with normal diet controls, and an inhibition of p38 phosphorylation in liver of Compound 2 treated mice. ND-V: Normal diet-Vehicle. HFD-V: High fat diet-Vehicle. HFD-SB 30: High fat diet-Compound 2 administered at 30 mg/kg.

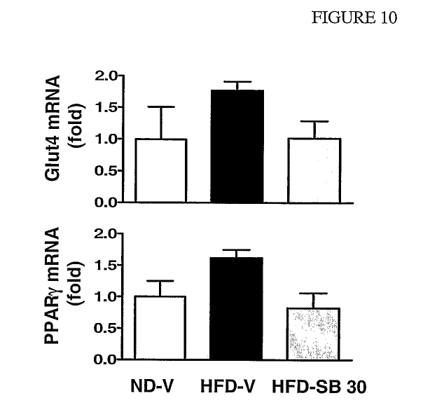

FIG. 10 demonstrates gene expression analysis showed that C57BL/6 mice on high fat diet for 14-15 weeks had increased GLUT4 and PPARγ mRNA expression in adipose tissue. Compound 2 treatment decreased adipose PPARγ and GLUT4 mRNA expression. ND-V: Normal diet-Vehicle. HFD-V: High fat diet-Vehicle. HFD-SB 30: High fat diet-Compound 2 administered at 30 mg/kg.

### SUMMARY OF THE INVENTION

The present invention is directed to the novel use of treating obesity in a mammal in need thereof comprising administering to said mammal an effective amount of a p38 kinase inhibitor.

Another embodiment of the invention is directed to a method of reducing body weight in a mammal in need thereof comprising administering to said mammal an effective amount of a p38 kinase inhibitor.

Another embodiment of the invention is directed to a method of reducing body mass in a mammal in need thereof comprising administering to said mammal an effective amount of a p38 kinase inhibitor.

### DESCRIPTION OF THE INVENTION

The present invention relates to the prevention and treatment of obesity. More particularly, this invention relates to a method of a) treating, preventing, suppressing, inhibiting, or reducing obesity; b) promoting, increasing or facilitating weight loss; or c) altering the body composition.

In one embodiment, this invention relates to a method of treating a mammal, preferably a human, suffering from obesity comprising the step of administering to the subject a p38 kinase inhibitor, in an amount effective to treat obesity in the mammal.

In another embodiment, this invention relates to a method of preventing, suppressing, inhibiting or reducing the incidence of obesity in a mammal, preferably a human, comprising the step of administering to the mammal a p38 kinase inhibitor in an amount effective to prevent, suppress, inhibit or reduce the incidence of obesity in the mammal

In another embodiment, this invention relates to a method of promoting, increasing or facilating weight loss in a mammal, prefereably a human, comprising the step of administering to the mammal as a p38 kinase inhibitor in an amount effective to promote, increase or facilitate weight loss in the mammal.

Role of p38 in Adipogenesis:

Adipogenesis involves the differentiation of adipocytes and accumulation of lipids, under tight control of gene transcription by hormones and associated transcription factors and signaling pathways. Activation of p38 is observed in multiple fibroblast cell lines undergoing adipogenesis. Constitutively active MKK6 or salicylate, leading to activation of p38, induces spontaneous 3T3-L1 adipogenesis (See Engelman et al., J Biol Chem 1999, 274:35630-8), which can be blocked by specific inhibitors of p38 (Engelman et al., J Biol Chem 1998, 273:32111-20; and Takenouchi et al., Cell Biology International. 2004, 28:209-16). In addition, glucose transporter expression and activity in muscle and adipose tissue (See Fujishiro et al., Supra, Huang et al., Supra and Somwar et al., Supra) is p38 dependent. Using a dominant-negative p38 mutant and p38 inhibitors (Compound 1,4-(4-Fluorophenyl)-2-(4-methylsulfinylphenyl)-5-(4-pyridyl)imidazole and azaazulene pharmacophores A291077 and A304000, Somwar et al. reported that p38 reduced insulin-induced glucose uptake in 3T3-L1 adipocytes by reducing GLUT4 activity via direct inhibition of p38. The p38-dependent glucose uptake in adipocytes and glyconeogenesis in liver therefore provides the rationale for use of a p38 inhibitor in obese patients. The p38 kinase was not activated upon insulin stimulation in adipocytes from either healthy or diabetic subjects, but a higher basal level of p38 activation was observed in diabetic subjects [Carlson et al., supra]. Similarly, Gum R J et al. Supra, reported that phosphorylation of p38 was increased in liver of ob/ob mice (a strain prone to obesity and diabetes) relative to lean litter mates, further demonstrating a role of p38 in adipogenesis and hence, use of a p38 inhibitor in the treatment of obesity.

Role of p38 in Glucose Metabolism/Diabetes

Type 2 diabetes arises from a combination of impaired insulin action and defective pancreatic β-cell function. The combination of impaired insulin-dependent glucose metabolism in skeletal muscle and impaired β-cell function causes an increase of hepatic glucose production, leading to multiple tissue abnormalities (Cline et al., N Engl J Med 1999, 341: 240-246; Chen et al., J Clin Endocrinol Metab 1987, 64: 17-21; and DeFronzo et al., Diabetes 1998, 37: 667-687)

Glucose production in liver is accomplished by glycogenolysis and glyconeogenesis. Hepatic gluconeogenesis plays an essential role in maintaining plasma glucose during physiological fasting and is a major contributor to fasting and postprandial hyperglycemia in both type 1 and type 2 diabetes (Pilkis et al., Annu Rev Physiol 1992, 54: 885-909; and Nordlie et al., Annu Rev Nutr 1999, 19: 379-406). High levels of p38 phosphorylation have been observed in adipose tissue of type 2 diabetic patients and in liver of ob/ob mice. Blockade of p38 by either a chemical inhibitor or siRNA diminished fasting plasma glucose levels significantly in normal and diabetic mice and reduced gluconeogenesis in primary hepatocytes and liver by blocking expression of key gluconeogenic enzymes. [Cao et al, Mol Cell Biol. 2004 24(7):3057-67; Collin et al., Supra]. Also, p38 inhibition can block fasting-induced phosphorylation and expression of the PPARγ coactivator 1α(PGC-1α) gene and phosphorylation of cAMP response element binding protein (CREB) in liver, two nuclear factors that are key components in the control of gluconeogenesis.

The models described here, are diet induced obesity and insulin resistance similar to that observed in Type 2 diabetes. A representative p38 inhibitor compound was used and was found effective in reducing body weight and fat mass under several different conditions, supporting the utility of a p38 inhibitor in obesity and obesity leading to additional inflammatory conditions.

Therefore, as the p38 kinase and various components of the p38 signaling pathway have been found to be implicated in metabolic syndrome, a precursor condition linking cardiovascular disease, obesity, diabetes and low-level inflammation, one aspect of the present invention is the use of a p38 inhibitor for treatment of obesity and weight loss therapy.

Therefore, therapeutic control of obesity is seen as critically important in the clinical management and treatment of diabetes mellitus.

Accordingly, the present invention provides for a method of treating a cytokine-mediated disease, obesity and reduction of body mass, or fat mass, which comprises administering to a mammal, preferably a human, in need thereof an effective cytokine-interfering amount of a compound which is an inhibitor of the CSBP/p38 kinase or of the signalling pathway of CSBP/p38 kinase.

As used herein, the term “cytokine” refers to any secreted polypeptide that affects the functions of cells and is a molecule which modulates interactions between cells in the immune, inflammatory or hematopoietic response. A cytokine includes, but is not limited to, monokines and lymphokines, regardless of which cells produce them. For instance, a monokine is generally referred to as being produced and secreted by a mononuclear cell, such as a macrophage and/or monocyte. Many other cells however also produce monokines, such as natural killer cells, fibroblasts, basophils, neutrophils, endothelial cells, brain astrocytes, bone marrow stromal cells, epideral keratinocytes and B-lymphocytes. Lymphokines are generally referred to as being produced by lymphocyte cells. Examples of cytokines include, but are not limited to, Interleukin-1 (IL-1), Interleukin-6 (IL-6), Interleukin-8 (IL-8), Tumor Necrosis Factor-alpha (TNF-α) and Tumor Necrosis Factor beta (TNF-β).

As used herein, the term “cytokine interfering” or “cytokine suppressive amount” refers to an effective amount of a compound which is a p38 inhibitor which will cause a decrease in the in vivo levels of the cytokine to normal or sub-normal levels, when given to a patient for the prophylaxis or treatment of a disease state which is exacerbated by, or caused by, excessive or unregulated cytokine production.

The p38 inhibitor compounds of may be administered in conventional dosage forms prepared by combining a p38 inhibitor compound with standard pharmaceutical carriers according to conventional procedures. The p38 inhibitor compounds may also be administered in conventional dosages in combination with a known, second therapeutically active compound. These procedures may involve mixing, granulating and compressing or dissolving the ingredients as appropriate to the desired preparation. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof.

The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time delay material well known to the art, such as glyceryl mono-stearate or glyceryl distearate alone or with a wax.

A wide variety of pharmaceutical forms can be employed. Thus, if a solid carrier is used, the preparation can be tableted, placed in a hard gelatin capsule in powder or pellet form or in the form of a troche or lozenge. The amount of solid carrier will vary widely but preferably will be from about 25 mg to about 1 g. When a liquid carrier is used, the preparation will be in the form of a syrup, emulsion, soft gelatin capsule, sterile injectable liquid such as an ampule or as a nonaqueous liquid suspension.

The p38 inhibitory compounds may be administered topically, that is by non-systemic administration. This includes the application of a p38 inhibitor compound externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

For all methods of use disclosed herein the daily oral dosage regimen of a p38 inhibitor compound will be from about 0.05 to about 80 mg/kg of total body weight, preferably from about 0.1 to 30 mg/kg, more preferably from about 0.5 mg to 15 mg/kg, administered in one or more daily doses. The daily parenteral dosage regimen about 0.1 to about 80 mg/kg of total body weight, preferably from about 0.2 to about 30 mg/kg, and more preferably from about 0.5 mg to 15 mg/kg, administered in one or more daily doses. The daily topical dosage regimen will preferably be from 0.01 mg to 150 mg, administered one to four times daily. The daily inhalation dosage regimen will be from about 0.05 microgram/kg to about 1 mg/kg per day, preferably from about 0.2 microgram/kg to about 20 microgram/kg, administered in one or more daily doses.

It will also be recognized by one of skill in the art that the optimal quantity and spacing of individual dosages of a p38 inhibitor compound will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a compound given per day for a defined number of days, can be ascertained by those skilled in the art using conventional course of treatment determination tests.

The p38 inhibitory kinase compounds and pharmaceutical formulations thereof, may be used in combination with, or include one or more other therapeutic agents, for example selected from anti-inflammatory agents, anticholinergic agents (particularly an M₁, M₂, M₁/M₂ or M₃ receptor antagonist), β₂-adrenoreceptor agonists, antiinfective agents (e.g. antibiotics, antivirals), or antihistamines. Other compounds for use in combination with the p38 inhibitors are compounds such as (a) DP-IVinhibitors; (b) insulin sensitizers selected from the group consisting of (i) PPAR agonists and (ii) biguanides; (c) insulin and insulin mimetics; (d) sulfonylureas and other insulin secretagogues; (e) x-glucosidase inhibitors; (f) glucagon receptor antagonists; (g) GLP-1, GLP-1 mimetics, and GLP-1 receptor agonists; (h) GIP, GIP mimetics, and GIP receptor agonists; (i) PACAP, PACAP mimetics, and PACAP receptor agonists; (I) cholesterol lowering agents selected from the group consisting of (i) HMG-CoA reductase inhibitors, (ii) sequestrants, (iii) nicotinyl alcohol, nicotinic acid and salts thereof, (iv) PPARoc agonists, (v) PPARoc/y dual agonists, (vi) inhibitors of cholesterol absorption, (vii) acyl CoA:cholesterol acyltransferase inhibitors, and (viii) anti oxidants; (k) PPARo agonists; (l) antiobesity compounds; (m) an ileal bile acid transporter inhibitor (n) anti-inflammatory agents excluding glucocorticoids; and (o) protein tyrosine phosphatase-1B (PTP-1B) inhibitors, said compounds being administered to the patient in an amount that is effective to treat the desired condition.

Another aspect of the invention is a pharmaceutical composition is disclosed which comprises (1) a p38 inhibitor compound and (2) a compound selected from the group consisting of: (a) DP-IV inhibitors; (b) insulin sensitizers selected from the group consisting of (i) PPAR agonists and (ii) biguanides; (c) insulin and insulin mimetics; 35 (d) sulfonylureas and other insulin secretagogues; (e) oc-glucosidase inhibitors; (I) glucagon receptor antagonists; (g) GLP-1, GLP-1 mimetics, and GLP-1 receptor agonists; (h) GIP, GIP mimetics, and GIP receptor agonists; (i) PACAP, PACAP mimetics, and PACAP receptor 3 agonists; (I) cholesterol lowering agents selected from the group consisting of (i) HMG-CoA reductase inhibitors, (ii) sequestrants, (iii) nicotinyl alcohol, nicotinic acid or a salt thereof, (iv) PPARoc agonists, (v) PPARcc/y dual agonists, (vi) inhibitors of cholesterol absorption, (vii) acyl CoA:cholesterol acyltransferase 10 inhibitors, and (viii) anti-oxidants; (k) PPARo agonists; (l) antiobesity compounds; (m) an ileal bile acid transporter inhibitor; (n) anti-inflammatory agents other than glucocorticoids; and (o) protein tyrosine phosphatase-1B (PTP-1B) inhibitors; and (3) a pharmaceutically acceptable carrier or diluent.

Examples of other active ingredients that may be administered in combination with a p38 inhibitor compound and either administered separately or in the same pharmaceutical composition, include, but are not limited to: (a) dipeptidyl peptidase IV (DP-IV) inhibitors; (b) insulin sensitizers including (i) PPARy agonists such as the glitazones (e.g. troglitazone, pioglitazone, englitazone, MCC-555, rosiglitazone, and the like) and other PPAR ligands, including PPARoc/′ dual agonists, such as KRP-25 297, and PPARoc agonists such as gemfibrozil, clofibrate, fenofibrate and bezafibrate, and (ii) biguanides, such as metformin and phenformin; (c) insulin or insulin mimetics; (d) sulfonylureas and other insulin secretagogues such as tolbutamide S and glipizide, meglitinide and related materials; (e) oc-glucosidase inhibitors (such as acarbose); (f) glucagon receptor antagonists such as those disclosed in WO 98/04528, WO 99/01423, WO 00/39088 and WO 00/69810; (g) GLP-1, GLP-1 mimetics, and GLP-1 receptor agonists such as 10 those disclosed in WO 2000/42026 and WO 2000/59887; (h) GIP, GIP mimetics such as those disclosed in WO 2000/58360, and GIP receptor agonists; (i) PACAP, PACAP mimetics, and PACAP receptor 3 agonists such as those disclosed in WO 2001/23420; (I) cholesterol lowering agents such as (i):G-CoA reductase inhibitors (lovastatin, simvastatin, pravastatin, fluyastatin, atorvastatin, rivastatin, itavastatin, rosuvastatin, and other stating), (ii) sequestrants (cholestyramine, colestipol, and dialkylaminoalkyl derivatives of a cross-linked dextran), (iii) nicotinyl alcohol, nicotinic acid or a salt thereof, (iv) inhibitors of cholesterol absorption, such as for example ezetimibe and beta-sitosterol, (v) acyl CoA:cholesterol acyltransferase inhibitors, such as for example avasimibe, and (vi) anti-oxidants such as probucol; (k) PPAR agonists, such as those disclosed in WO97/28149; (1) antiobesity compounds such as fenfluramine, dexfenfluramine, phentermine, sibutramine, orlistat, neuropeptide Y5 inhibitors, CB 1 receptor inverse agonists and antagonists, and p3 adrenergic receptor agonists; (m) an ileal bile acid transporter inhibitor; (n) agents intended for use in inflammatory conditions other than glucocorticoids, such as aspirin, non-steroidal anti-inflammatory drugs, azulfidine, and cyclooxygenase 2 selective inhibitors, and (o) protein tyrosine phosphatase-1B (PTP-1B) inhibitors.

It will be clear to a person skilled in the art that, where appropriate, the other therapeutic ingredient(s) may be used in the form of salts, (e.g. as alkali metal or amine salts or as acid addition salts), or prodrugs, or as esters (e.g. lower alkyl esters), or as solvates (e.g. hydrates) to optimise the activity and/or stability and/or physical characteristics (e.g. solubility) of the therapeutic ingredient. It will be clear also that where appropriate, the therapeutic ingredients may be used in optically pure form.

Suitable p38 or Cytokine Suppressive Anti-Inflammatory Drug (CSAID) compounds are well known in the art, and an assay for determining CBSP/p38 inhibition is also readily available using assays disclosed in the below noted patents or applications. Representative compounds which are inhibitors of p38 may be found in:
WO 05/073219; WO 05/073189; WO 05/073217; WO 05/07232; 2005/0038014, Angell et al.; 2004/0266839, Angell et al.; 2004/0249161, Angell et al.; 2005/0020540, Angell et al.; 2005/0176964, Aston et al.; 2005/0065195, Angell et al.; 2005/0090491, Angell et al.; 2004/0242868, Angell et al.; 2004/0267012, Angell et al.; WO 03/093248, Angell et al; WO 03/032970, Angell et al.; WO 05/014550, Walker, A.; WO 04/010995, Angell et al., WO 04/089875, Aston, N.; WO 04/089876, Aston, N.; WO 04/089874, Aston, et al.; U.S. Pat. No. 5,716,972; U.S. Pat. No. 5,686,455; U.S. Pat. No. 5,656,644; U.S. Pat. No. 5,916,891; U.S. Pat. No. 5,593,992; U.S. Pat. No. 6,288,062; U.S. Pat. No. 5,545,669; U.S. Pat. No. 5,559,137; U.S. Pat. No. 5,998,425; WO 96/21654; U.S. Pat. No. 5,658,903; U.S. Pat. No. 6,369,068; U.S. Pat. No. 5,739,143; U.S. Pat. No. 5,716,955; U.S. Pat. No. 6,372,741; U.S. Pat. No. 6,096,748; U.S. Pat. No. 6,414,150; U.S. Pat. No. 5,774,127; U.S. Pat. No. 6,329,526; U.S. Pat. No. 5,929,076; U.S. Pat. No. 5,756,499; U.S. Pat. No. 6,046,08; U.S. Pat. No. 6,489,325; U.S. Pat. No. 6,610,695; U.S. Pat. No. 6,362,193; U.S. Pat. No. 6,548,520; U.S. Pat. No. 6,589,954; US 2004/097493; US 004/209886; US 2004/09903; US 2004/09904; US 2004/54236; WO 99/61440; U.S. Pat. No. 6,649,617; U.S. Pat. No. 6,548,503; U.S. Pat. No. 6,469,018; U.S. Pat. No. 6,509,363; U.S. Pat. No. 6,809,199; U.S. Pat. No. 6,962,996; U.S. Pat. No. 6,800,626; 6,509,363, U.S. Pat. No. 6,759,535; WO 01/38314; WO 01/38313; WO 01/38312; U.S. Pat. No. 6,759,410; WO 2001/64679; WO 2002/059083; WO 2002/32862; WO 2002/060869; WO 2001/37835; WO 2002/39954; WO 2004/021979, now U.S. Pat. No. 6,809,199; WO 2003/088972; WO 2004/073628; WO 2005/123744; WO 2003/033502; WO 2003/045941; WO 2004/000846; WO 2004/014920; WO 2004/031188; WO 2004/113348; WO 2004/113347; WO 2003/087087; WO 2004/004720; WO 2004/089929; WO 2004/100946; WO 2004/078116; WO 2004/078747; WO 2004/102636; U.S. Pat. No. 6,344,476; WO 2003/068299; WO 2003/068746; WO 2003/008413; WO 2003/076405; WO 99/32463; US 2002/065296; US 2004/102636; WO 2003/064417; WO 2003/064418; WO 2003/064419; WO 2003/087096; WO 2004/004725; WO 2004/014870; WO 2004/014387; WO 2004/043367; WO 2004/02956; WO 2004/099156; WO 2002/040486; WO 2002/096426; WO 2003/002544; WO 2003/024899; WO 2003/053941; WO 2003/090912; WO 2003/091229; WO 2003/099820; WO 2004/060306; WO 2004/078756; WO 004/013139; WO 2004/014900; WO 2003/074530; WO 2002/018379; WO 2002/064594; WO 2001/029042; WO 2003/082871; WO 2004/014907; WO 2003/020715; WO 2002/058695; WO 2003/000682; WO 2003/097062; WO 2004/101529; WO 2002/072576; WO 2004/087615; WO 2004/143119; WO 2004/077682; WO 2004/0092547; WO 2004/157877; WO 2002/072579; WO 2004/020438; WO 2004/020440; WO 2004/072072; WO 2004/058176; WO 2004/087074; WO 2003/026663; WO 2003/059891; WO 2003/068230; WO 2002/044168; WO 2002/046158; WO 2003/069248; WO 2002/042292; WO 2004/022712; WO 2004/032874; U.S. Pat. No. 6,476,031; WO 2002/100405; WO 2004/037814; WO 2004/072038; WO 2000/017204; WO 2000/017175; WO 2001/070695; WO 2002/092087; WO 2002/100405; WO 2000/26209; WO 2000/18738; WO 00/20402; WO 99/64400; WO 2000/01688; WO 2000/07980; WO 2000/07991; WO 00/06563; WO 00/12074; WO 2000/12497; WO 2000/31072; WO 2000/31063; WO 2000/23072; WO 2000/31065; WO 2000/35911; WO 2000/39116; WO 2000/43384; WO 2000/41698; WO 99/58502; WO 99/58523; WO 99/57101; WO 99/61426; WO 99/64400; WO 99/59960; WO 99/15164; WO 99/59959; WO 99/17204; WO 99/00357; WO 99/03837; WO 99/01441; WO 99/01449; WO 99/03484; WO 98/47892; WO 98/56377; WO 98/22109; WO 98/24782; WO 98/24780; WO 98/22457; WO 98/52558; WO 98/52941; WO 98/52937; WO 98/52940; WO 98/56788; WO 98/27098; WO 98/47899; WO 98/50356; WO 97/36587; WO 97/47618; WO 97/16442; WO 97/16441; WO 97/12876; WO 95/09853; WO 95/09851; WO 95/09847; WO 95/09852; WO 92/12154; WO 94/19350; Lee et al., Current Med Chem, Vol. 12, pp 2979-2994 (2005); Goldstein et al., J. Med Chem., Vol. 49, pgs 1562-1575 (2006), including RO-3201195 S-[5-amino-1-(4-fluorophenyl)-1H-pyrazol-4-yl]-[3-(2,3-dihydroxypropoxy)phenyl]-methanone; Doramapimod (Birb-796/Birb796BS), see also WO 2004/49742; Scio-469 and Scio-323; de Dios, A. et al., J. Med. Chem., Vol. 48, pp. 2270-2273 (2005); Kuliopulos et al, Throm Haemost., 92, 1387 (2004); PCT/US2006/010855, Attorney Docket No.: PU61314; PCT/US2006/010908, Attorney Docket No.: PU61335; PCT/US2006/010792, Attorney Docket No.: PU61344; whose disclosures are all incorporated herein by reference in their entirety.

One embodiment of the present invention is the use of the p38 kinase inhibitors 8-(2,6-Difluoro-phenyl)-4-(4-fluoro-2-methyl-phenyl)-2-(2-hydroxy-1-hydroxymethyl-ethylamino)-8H-pyrido[2,3-d]pyrimidin-7-one, or a pharmaceutically acceptable salt thereof, or 6-(5-cyclopropylcarbamoyl-3-fluoro-2-methyl-phenyl)-N-(2,2-dimethylpropyl)-nicotinamide, or a pharmaceutically acceptable salt thereof, in the methods disclosed herein.

Another embodiment of the present invention is the use of a compound of the formula disclosed in WO 2004/072038, and in particular the compound which is:

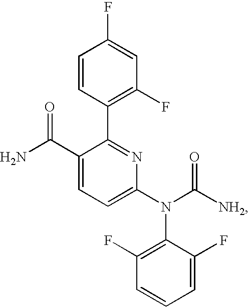

or a pharmaceutically acceptable salt thereof (alternatively referred to as VX-702).

### Biological Experimentation

A representative compound which is an inhibitor of the p38 kinase was used in the biological examples shown below. That compound is trans-1-(4-hydroxycyclohexyl)-4-(4-fluorophenyl)-5-[(2-methoxy)pyrimidin-4-yl]imidazole. This compound can be made as described in U.S. Pat. No. 6,251,914 whose disclosure is incorporated by reference herein.

One aspect of the invention was to determine the effect of a representative p38 inhibitor, Compound 2 on body weight, fat mass, insulin level, and blood glucose in diet-AKR mice.

Experiment 1

Compound 2 was administered for four weeks treatment in 18-19 weeks old male, diet-induced obese (DIO) AKR mice.

Methods: Male AKR mice, from Jackson Laboratories, group housed, 18-19 weeks old, on high fat diet (HFD, D12331 rodent diet, 58 kcal % from fat and sucrose. Research Diets, Inc., New Brunswick, N.J.) started at 4-5 weeks old. A parallel group of mice on normal diet (ND, Purina 5001) was served as diet control. During the last 4 weeks of diet period, mice on HFD were treated with vehicle (0.5% tragacanth in 0.03N HCl), Compound 2 was administered at 3 mg/kg, and 10 mg/kg, and water (control for vehicle), twice daily (BID), orally (PO) at 10 ml/kg. Mice on normal diet (ND) were treated with vehicle. Mouse number in each group was 7-10. Body weight (BW) was monitored once a week. Body composition was monitored every other week by using EchoMRI™ Whole Body Composition Analyzer (Echo Medical Systems, Houston, Tex.). Fasting blood glucose was measured at 2 weeks of treatment. Postprandial blood glucose was measured at before and end of treatment. Fasting serum insulin was measured at dosing 2 weeks via orbital bleeding. Serum IL-6 was measured at the end of treatment under fasting condition. Insulin tolerance test (ITT) was also performed at end of the diet period.

Results: Male AKR mice on high fat diet for 14-15 weeks had a stable weight 40% greater than normal chow controls with a doubling in the fat mass (FIGS. 1 and 2). Mice on HFD were insulin resistant as indicated by elevated postprandial glucose (14835 5 vs 132±4 mg/dL, p<0.05), fasting blood glucose (162±14 vs 111±5 mg/dL, p<0.01), fasting insulin (0.45±0.02 vs 0.18±0.02 ng/ml, p<0.01) levels, and decreased response during ITT (FIG. 3). Treatment of AKR mice with Compound 2, at 3 & 10 mg/kg, PO, BID, for the last 4 weeks of 14 weeks on HFD resulted in a 4-9% reduction in body weight (BW, p<0.05) and a trend towards reduced fat mass (FIGS. 1 and 2), and had no effect on lean mass. The treatment reduced serum inflammatory cytokine IL-6 as expected (FIG. 4). Treatment with Compound 2 had no effect on blood glucose. DIO mice treated with water showed similar outcome as that treated with vehicle (FIGS. 1 and 2). Thus the vehicle did not cause any change on the parameters.

Experiment 2

Compound 2 was administered for a two week treatment in 24-25 weeks old male DIO AKR mice (older).

Methods: Male AKR mice, from Jackson Laboratories, group housed, 24-25 weeks old, on high fat diet (HFD, D12331 rodent diet, 58 kcal % from fat and sucrose. Research Diets, Inc., New Brunswick, N.J.) started at 4-5 weeks old. A parallel group of mice on normal diet (ND, Purina 5001) was served as diet control. During the last 2 weeks of diet period, mice on HFD were treated with vehicle (0.5% tragacanth in 0.03N HCl), Compound 2 administered at 10, and 30 mg/kg, BID, PO (10 ml/kg). Mice on ND were treated with vehicle. Mouse number in each group was 7-10. Body weight (BW), body composition, postprandial blood glucose, and fasting serum insulin were measured.

Results: Male AKR mice on high fat diet for 20-21 weeks had a stable weight 40% greater than normal chow controls (51.7±0.5 vs 34.5±0.8 g. p<0.01) with a doubling in the fat mass (FIGS. 5 and 6). Mice on HFD were insulin resistant as indicated by elevated postprandial blood glucose (180±11 vs 131±2 mg/dL, p<0.01), and fasting insulin (1.26±0.22 vs 0.55±0.14 ng/ml, p<0.05) levels. Treatment of AKR mice with Compound 2 administered at 10 & 30 mg/kg, PO, BID, for the last 2 weeks of 20 weeks on HFD resulted in a 4-9% reduction in body weight (BW, p<0.05), and a marked reduce in fat mass (FIGS. 5 and 6). The effect of BW and fat mass appeared after 4 days of treatment, and became more pronounced over the 2 weeks of treatment. Treatment with Compound 2 had no effect on blood glucose.

Experiment 3

The effects of a p38 inhibitor, Compound 2 was determined on body weight, fat mass, insulin level, blood glucose, p38 phosphorylation in liver, and gene of interested in adipose tissue in the male DIO C57/B6 mice.

Methods: Male C57BL/6 mice, from Jackson Laboratories, group housed, 18-19 weeks old, on high fat diet (HFD, D12331 rodent diet, 58 kcal % from fat and sucrose. Research Diets, Inc., New Brunswick, N.J.) started at 4-5 weeks old. A parallel group of mice on normal diet (ND, Purina 5001) was served as diet control. During the last 4 weeks of diet period, mice on HFD were treated with vehicle (0.5% tragacanth in 0.03N HCl), Compound 2 administered at 3, 10, and 30 mg/kg, BID, PO (10 ml/kg). Mice on ND were treated with vehicle. Mouse number in each group was 8-10. Body weight (BW), body composition, fasting blood glucose, postprandial blood glucose and insulin were measured. At the end of treatment, liver was collected to determine p38 phosphorylation. Epididymal fat pad was collected to determine PPARγ and GLUT4 gene expression.

Results: Male C57BL/6 mice on high fat diet for 14-15 weeks had a stable weight 20+% greater than normal chow controls (32.6±0.9 vs 27.0±0.5 g. p<0.01) with a doubling in the fat mass (FIGS. 7 and 8). Mice on HFD were insulin resistant as indicated by elevated postprandial blood glucose (135±6 vs 120±6 mg/dL, p<0.05), fasting blood glucose (51±2 vs 48±1 mg/dL, p<0.05) and fasting insulin (1.68±1.02 vs 0.40±0.12 ng/ml, p<0.05) levels. Treatment of AKR mice with Compound 2 administered at 30 mg/kg, PO, BID, for the last 4 weeks of 14 weeks on HFD resulted in a significant reduction in body weight (p<0.05), and a marked reduce in fat mass (FIGS. 7 and 8). Treatment with Compound 2 had no effect on lean mass and blood glucose. Western analysis demonstrated an inhibition of p38 phosphorylation in the liver of Compound 2 treated mice (FIG. 9). Gene expression analysis showed decreased PPARγ and GLUT4 expression in adipose of the treated mice (FIG. 10).

### Summary of Experimental Data

Male AKR and C57BL/6 mice on the HFD for 14 to 22 weeks reached a stable weight ˜40% greater than normal chow controls with a doubling in the fat mass. Mice on HFD were insulin resistant as indicated by elevated fasting and postprandial glucose and insulin levels as well as a decreased response to an insulin tolerance test. Treatment of AKR mice with Compound 2 administered at 3 & 10 mg/kg, PO, BID, for the last 4 weeks of 14 weeks on HFD resulted in a 4-9% reduction in body weight (BW, p<0.05) and a trend towards reduced fat mass. The treatment reduced serum inflammatory cytokine IL-6, as expected. Treatment of AKR mice with Compound 2 administered at 10 & 30 mg/kg, PO, BID, for the final 2 of 22 weeks on HFD also decreased BW and significantly reduced fat mass (p<0.05). C57 mice treated with Compound 2 administered at 3-30 mg/kg, PO, BID, for the final 4 weeks of 15 weeks on HFD had significant reductions in both BW and fat mass (p<0.01). Treatment with Compound 2 did not affect lean mass in either AKR or C57 mice, and did not have any significant effect on blood glucose or response to an ITT. Western analysis demonstrated an inhibition of p38 phosphorylation in liver of Compound 2 treated mice. Gene expression analysis showed decreased PPARγ and GLUT4 expression in adipose of the treated mice.

Treatment with a representative p38 inhibitor, Compound 2, showed a reduced BW and fat mass in HFD obese mice while having a minimal effect on insulin resistance. It is hypothesized that the reduction in fat mass may be due to inhibition of PPARγ-mediated adipogenesis and GLUT4-mediated glucose uptake in adipose.

The cytokine-inhibiting effects of compounds may be determined by the following in vitro assays:

Fluorescence Anisotropy Kinase Binding Assay—Standard Volume

The kinase enzyme, fluorescent ligand and a variable concentration of test compound are incubated together to reach thermodynamic equilibrium under conditions such that in the absence of test compound the fluorescent ligand is significantly (>50%) enzyme bound and in the presence of a sufficient concentration (>10×Kᵢ) of a potent inhibitor the anisotropy of the unbound fluorescent ligand is measurably different from the bound value.

The concentration of kinase enzyme should preferably be ≧2×K_{f}. The concentration of fluorescent ligand required will depend on the instrumentation used, and the fluorescent and physicochemical properties. The concentration used must be lower than the concentration of kinase enzyme, and preferably less than half the kinase enzyme concentration.

The fluorescent ligand is the following compound:

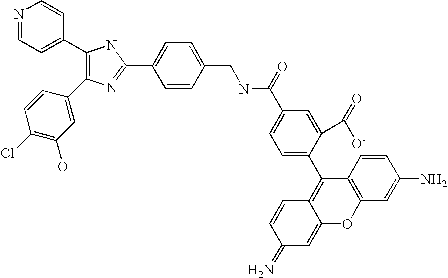

which is derived from 5-[2-(4-aminomethylphenyl)-5-pyridin-4-yl-1H-imidazol-4-yl]-2-chlorophenol and rhodamine green.

Recombinant human p38α is expressed as a GST-tagged protein. To activate this protein, 3.5 μM unactivated p38α is incubated in 50 mM Tris-HCl pH 7.5, 0.1 mM EGTA, 0.1% 2-mercaptoethanol, 0.1 mM sodium vanadate, 10 mM MgAc, 0.1 mM ATP with 200 nM MBP-MKK6 DD at 30 degrees for 30 mins. Following activation p38α is re-purified and the activity assessed using a standard filter-binding assay.

Protocol: All components are dissolved in buffer of composition 62.5 mM HEPES, pH 7.5, 1.25 mM CHAPS, 1 mM DTT, 12.5 mM MgCl₂ with final concentrations of 12 nM p38α and 5 nM fluorescent ligand. 30 μl of this reaction mixture is added to wells containing 1 μl of various concentrations of test compound (0.28 nM-16.6 μM final) or DMSO vehicle (3% final) in NUNC 384 well black microtitre plate and equilibrated for 30-60 mins at room temperature. Fluorescence anisotropy is read in Molecular Devices Acquest (excitation 485 nm/emission 535 nm).

Definitions: Ki=dissociation constant for inhibitor binding

K_{f}=dissociation constant for fluorescent ligand binding

Fluorescence Anisotropy Kinase Binding Low Volume Assay

The kinase enzyme, fluorescent ligand and a variable concentration of test compound are incubated together to reach thermodynamic equilibrium under conditions such that in the absence of test compound the fluorescent ligand is significantly (>50%) enzyme bound and in the presence of a sufficient concentration (>10×Ki) of a potent inhibitor the anisotropy of the unbound fluorescent ligand is measurably different from the bound value.

The concentration of kinase enzyme should preferably be 2×Kf. The concentration of fluorescent ligand required will depend on the instrumentation used, and the fluorescent and physicochemical properties. The concentration used must be lower than the concentration of kinase enzyme, and preferably less than half the kinase enzyme concentration.

The fluorescent ligand is the following compound:

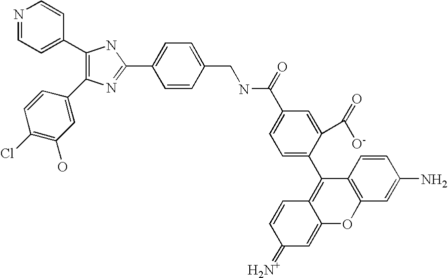

which is derived from 5-[2-(4-aminomethylphenyl)-5-pyridin-4-yl-1H-imidazol-4-yl]-2-chlorophenol and rhodamine green.

Recombinant human p38α is expressed as a GST-tagged protein. To activate this protein, 3.5 μM unactivated p38α is incubated in 50 mM Tris-HCl pH 7.5, 0.1 mM EGTA, 0.1% 2-mercaptoethanol, 0.1 mM sodium vanadate, 10 mM MgAc, 0.1 mM ATP with 200 nM MBP-MKK6 DD at 30 degrees for 30 mins. Following activation p38α is re-purified and the activity assessed using a standard filter-binding assay.

Protocol: All components are dissolved in buffer of composition 62.5 mM HEPES, pH 7.5, 1.25 mM CHAPS, 1 mM DTT, 12.5 mM MgCl₂ with final concentrations of 12 nM p38α and 5 nM fluorescent ligand. 30 μl of this reaction mixture is added to wells containing 0.1 μl of various concentrations of test compound (0.02 nM-25 μM final) or DMSO vehicle (1.7% final) in Greiner low volume 384 well black microtitre plate and equilibrated for 30-60 mins at room temperature. Fluorescence anisotropy is read in Molecular Devices Acquest (excitation 485 nm/emission 535 nm).

Definitions: Ki=dissociation constant for inhibitor binding

Kf=dissociation constant for fluorescent ligand binding

It is noted that there are two assay formats shown above for the Fluorescence anisotropy kinase binding assay. The only difference between these two assays is the volume used and the plate type. It has been demonstrated that there is no difference in potency between the two formats, and that the assays are considered to be equivalent.

Compounds are considered active in these assay's if they demonstrate a pIC50 of greater than 4.6 up to about a pIC50 of 9.0.

### Tr-Fret Assay

Time-Resolved Fluorescence Resonance Energy Transfer Kinase Standard Assay

Recombinant human p380: is expressed as a His-tagged protein. To activate this protein, 3 μM unactivated p38α is incubated in 200 mM Hepes pH 7.4, 625 mM NaCl, 1 mM DTT with 27 nM active MKK6 (Upstate), 1 mM ATP and 10 mM MgCl₂. The activity of the MKK6-activated p38α is assessed using a time-resolved fluorescence resonance energy transfer (TR-FRET) assay.

Biotinylated-GST-ATF2 (residues 19-96, 400 nM final), ATP (125M final) and MgCl2 (5 mM final) in assay buffer (40 mM HEPES pH 7.4, 1 mM DTT) are added to wells containing 1 ul of various concentrations of compound or DMSO vehicle (3% final) in NUNC 384 well black plate. The reaction is initiated by the addition of MKK6-activated p38 (100 pM final) to give a total volume of 30 ul. The reaction is incubated for 120 minutes at room temperature, then terminated by the addition of 15 μl of 100 mM EDTA pH 7.4. Detection reagent (15 μl) in buffer (100 mM HEPES pH 7.4, 150 mM NaCl, 0.1% w/v BSA, 1 mM DTT) containing antiphosphothreonine-ATF2-71 polyclonal antibody (Cell Signalling Technology, Beverly Mass., USA) labelled with W-1024 europium chelate (Wallac OY, Turku, Finland), and APC-labelled streptavidin (Prozyme, San Leandro, Calif., USA) is added and the reaction is further incubated for 60 minutes at room temperature. The degree of phosphorylation of GST-ATF2 is measured using a Packard Discovery plate reader (Perkin-Elmer Life Sciences, Pangbourne, UK) as a ratio of specific 665 nm energy transfer signal to reference europium 620 nm signal.

Time-Resolved Fluorescence Resonance Energy Transfer Kinase Low Volume Assay

Recombinant human p380: is expressed as a His-tagged protein. To activate this protein, 3 μM unactivated p38α is incubated in 200 mM Hepes pH7.4, 625 mM NaCl, 1 mM DTT with 27 nM active MKK6 (Upstate), 1 mM ATP and 10 MM MgCl₂. The activity of the MKK6-activated p38α is assessed using a time-resolved fluorescence resonance energy transfer (TR-FRET) assay.

Biotinylated-GST-ATF2 (residues 19-96, 400 nM final), ATP (125 μM final) and MgCl₂ (5 mM final) in assay buffer (40 mM HEPES pH 7.4, 1 mM DTT) are added to wells containing 0.1 μl of various concentrations of compound or DMSO vehicle (1.7% final) in Greiner low volume 384 well black plate. The reaction is initiated by the addition of MKK6-activated p38α (100 pM final) to give a total volume of 6 μl. The reaction is incubated for 120 minutes at room temperature, then terminated by the addition of 3 μl of detection reagent in buffer (100 mM HEPES pH 7.4, 150 mM NaCl, 0.1% w/v BSA, 1 mM DTT, 100 mM EDTA) containing antiphosphothreonine-ATF2-71 polyclonal antibody (Cell Signalling Technology, Beverly Mass., USA) labelled with W-1024 europium chelate (Wallac OY, Turku, Finland), and APC-labelled streptavidin (Prozyme, San Leandro, Calif., USA). The reaction is further incubated for 60 minutes at room temperature. The degree of phosphorylation of GST-ATF2 is measured using a BMG Rubystar plate reader (BMG, UK) as a ratio of specific 665 nm energy transfer signal to reference europium 620 nm signal.

It is noted that there are two assay formats shown above for the Time-resolved fluorescence resonance energy transfer kinase assay. The only difference between these two assays is the volume used and the plate type. It has been demonstrated that there is no difference in potency between the two formats, and that the assays are considered to be equivalent. Compounds are considered active in this assay if they demonstrate a pIC50 of greater than 4.6 up to about a pIC50 of greater than 10.0.

For purposes herein for the HTRF assay and the Fluorescence anisotropy kinase binding assay:

| pIC₅₀ | IC₅₀ (nM) | IC₅₀ (uM) |
|---|---|---|
| 4.00 | 100,000.0 | 100 |
| 5.00 | 100,000.0 | 10 |
| 6.00 | 1,000.0 | 1 |
| 7.00 | 100.0 | 0.1 |
| 8.00 | 10.0 | 0.01 |
| 9.00 | 1.0 | 0.001 |
| 10.00 | 0.1 | 0.0001 |

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

1. A method of treating obesity in a mammal in need thereof comprising the step of administering to said mammal an amount of a p38 kinase inhibitor effective to treat obesity.

2. The method according to claim 1 wherein the p38 kinase inhibitor is administered with a second therapeutic agent.

3. The method according to claim 1 wherein the p38 inhibitor and/or therapeutic agent is administered orally, topically (intranasal) or via inhalation (aerosol), or both topically and via inhalation.

4. The method according to claim 1 wherein the CSBP/p38 inhibitor is selected from a compound disclosed in WO 05/073219; WO 05/073189; WO 05/073217; WO 05/07232; 2005/0038014, Angell et al.; 2004/0266839, Angell et al.; 2004/0249161, Angell et al.; 2005/0020540, Angell et al.; 2005/0176964, Aston et al.; 2005/0065195, Angell et al.; 2005/0090491, Angell et al.; 2004/0242868, Angell et al.; 2004/0267012, Angell et al.; WO 03/093248, Angell et al.; WO 03/032970, Angell et al.; WO 05/014550, Walker, A.; WO 04/010995, Angell et al.; WO 04/089875, Aston, N.; WO 04/089876, Aston, N.; WO 04/089874, Aston, et al.; U.S. Pat. No. 5,716,972; U.S. Pat. No. 5,686,455; U.S. Pat. No. 5,656,644; U.S. Pat. No. 5,916,891; U.S. Pat. No.5,593,992; U.S. Pat. No. 6,288,062; U.S. Pat. No. 5,545,669; U.S. Pat. No. 5,559,137; U.S. Pat. No. 5,998,425; WO 96/21654; U.S. Pat. No. 5,658,903; U.S. Pat. No. 6,369,068; U.S. Pat. No. 5,739,143; U.S. Pat. No. 5,716,955; U.S. Pat. No. 6,372,741; U.S. Pat. No. 6,096,748; U.S. Pat. No. 6,414,150; U.S. Pat. No. 5,774,127; U.S. Pat. No. 6,329,526; U.S. Pat. No. 5,929,076; U.S. Pat. No. 5,756,499; U.S. Pat. No. 6,046,08; U.S. Pat. No. 6,509,363; U.S. Pat. No. 6,489,325; U.S. Pat. No. 6,610,695; U.S. Pat. No. 6,362,193; U.S. Pat. No. 6,548,520; U.S. Pat. No. 6,589,954; US 2004/097493; US 2004/209886; US 2004/09903; US 2004/09904; US 2004/54236; WO 99/61440; U.S. Pat. No. 6,649,617; U.S. Pat. No. 6,548,503; U.S. Pat. No. 6,469,018; U.S. Pat. No. 6,509,363; U.S. Pat. No. 6,809,199; U.S. Pat. No. 6,962,996; U.S. Pat. No. 6,800,626; U.S. Pat. No. 6,759,535; WO 01/38314; WO 01/38313; WO 01/38312; U.S. Pat. No. 6,759,410; WO 01/64679; WO 02/059083; WO 02/32862; WO 02/060869; WO 01/37835; WO 02/39954; WO 04/021979, U.S. Pat. No. 6,809,199; WO 03/088972; WO 04/073628; WO 2005/123744, WO 2003/033502; WO 2003/045941; WO 2004/000846; WO 2004/014920; WO 2004/031188; WO 2004/113348; WO 2004/113347; WO 2003/087087; WO 2004/004720; WO 2004/089929; WO 2004/100946; WO 2004/078116; WO 2004/078747; WO 2004/102636; U.S. Pat. No. 6,344,476; WO 2003/068299; WO 2003/068746; WO 2003/008413; WO 2003/076405; WO 99/32463; US 2002/065296; US 2004/102636; WO 2003/064417; WO 2003/064418; WO 2003/064419; WO 2003/087096; WO 2004/004725; WO 2004/014870; WO 2004/014387; WO 2004/043367; WO 2004/02956; WO 2004/099156; WO 2002/040486; WO 2002/096426; WO 2003/002544; WO 2003/024899; WO 2003/053941; WO 2003/090912; WO 2003/091229; WO 2003/099820; WO 2004/060306; WO 2004/078756; WO 2004/013139; WO 2004/014900; WO 2003/074530; WO 2002/018379; WO 2002/064594; WO 2001/029042; WO 2003/082871; WO 2004/014907; WO 2003/020715; WO 2002/058695; WO 2003/000682; WO 2003/097062; WO 2004/101529; WO 2002/072576; WO 2004/087615; WO 2004/143119; WO 2004/077682; WO 2004/0092547; WO 2004/157877; WO 2002/072579; WO 2004/020438; WO 2004/020440; WO 2004/072072; WO 2004/058176; WO 2004/087074; WO 2003/026663; WO 2003/059891; WO 2003/068230; WO 2002/044168; WO 2002/046158; WO 2003/069248; WO 2002/042292; WO 2004/022712; WO 2004/032874; U.S. Pat. No. 6,476,031; WO 2002/100405; WO 2004/037814; WO 2004/072038; WO 2000/017204; WO 2000/017175; WO 2001/070695; WO 2002/092087; WO 2002/100405; WO 2000/26209; WO 2000/18738; WO 00/20402; WO 99/64400; WO 2000/01688; WO 2000/07980; WO 2000/07991; WO 00/06563; WO 00/12074; WO 2000/12497; WO 2000/31072; WO 2000/31063; WO 2000/23072; WO 2000/31065; WO 2000/35911; WO 2000/39116; WO 2000/43384; WO 2000/41698; WO 99/58502; WO 99/58523; WO 99/57101; WO 99/61426; WO 99/64400; WO 99/59960; WO 99/15164; WO 99/59959; WO 99/17204; WO 99/00357; WO 99/03837; WO 99/01441; WO 99/01449; WO 99/03484; WO 98/47892; WO 98/56377; WO 98/22109; WO 98/24782; WO 98/24780; WO 98/22457; WO 98/52558; WO 98/52941; WO 98/52937; WO 98/52940; WO 98/56788; WO 98/27098; WO 98/47899; WO 98/50356; WO 97/36587; WO 97/47618; WO 97/16442; WO 97/16441; WO 97/12876; WO 95/09853; WO 95/09851; WO 95/09847; WO 95/09852; WO 92/12154; WO 94/19350; Lee et al., Current Med Chem, Vol. 12, pp 2979-2994 (2005); Goldstein et al., J. Med Chem., Vol. 49, pgs 1562-1575 (2006), RO-3201195=S-[5-amino-1-(4-fluorophenyl)-1H-pyrazol-4-yl]-[3-(2,3-dihydroxypropoxy)phenyl]-methanone; Doramapimod (Birb-796/Birb796BS), WO 2004/49742; Scio-469, Scio-323; or de Dios, A. et al., J. Med. Chem., Vol. 48, pp. 2270-2273 (2005).

5. The method according to claim 4 wherein the CSBP/p38 inhibitor is selected from a compound disclosed in WO 2004/072038; 2005/0176964; U.S. Pat. No. 6,509,363, WO 01/64679; or WO 02/059083.

6. The method according to claim 4 wherein the CSBP/p38 inhibitor is RO-3201195 (S-[5-amino-1-(4-fluorophenyl)-1H-pyrazol-4-yl]-[3-(2,3-dihydroxypropoxy)phenyl]-methanone); Doramapimod (Birb-796/Birb796BS); 8-(2,6-Difluoro-phenyl)-4-(4-fluoro-2-methyl-phenyl)-2-(2-hydroxy-1-hydroxymethyl-ethylamino)-8H-pyrido[2,3-d]pyrimidin-7-one, or a pharmaceutically acceptable salt thereof; 6-(5-cyclopropylcarbamoyl-3-fluoro-2-methyl-phenyl)-N-(2,2-dimethylpropyl)-nicotinamide, or a pharmaceutically acceptable salt thereof; Scio-469, Scio-323, or VX-702.

7. A method of reducing body weight in a mammal in need thereof comprising the step of administering to said mammal an amount of a p38 kinase inhibitor effective to reduce body weight.

8. The method according to claim 7 wherein the p38 kinase inhibitor is administered with a second therapeutic agent.

9. The method according to claim 7 wherein the p38 inhibitor and/or second therapeutic agent is administered orally, topically (intranasal) or via inhalation (aerosol), or both topically and via inhalation.

10. The method according to claim 7 wherein the CSBP/p38 inhibitor is selected from a compound disclosed in WO 05/073219; WO 05/073189; WO 05/073217; WO 05/07232; 2005/0038014, Angell et al.; 2004/0266839, Angell et al.; 2004/0249161, Angell et al.; 2005/0020540, Angell et al.; 2005/0176964, Aston et al.; 2005/0065195, Angell et al.; 2005/0090491, Angell et al.; 2004/0242868, Angell et al.; 2004/0267012, Angell et al.; WO 03/093248, Angell et al.; WO 03/032970, Angell et al.; WO 05/014550, Walker, A.; WO 04/010995, Angell et al.; WO 04/089875, Aston, N.; WO 04/089876, Aston, N.; WO 04/089874, Aston, et al.; U.S. Pat. No. 5,716,972; U.S. Pat. No. 5,686,455; U.S. Pat. No.5,656,644; U.S. Pat. No. 5,916,891; U.S. Pat. No.5,593,992; U.S. Pat. No. 6,288,062; U.S. Pat. No. 5,545,669; U.S. Pat. No. 5,559,137; U.S. Pat. No. 5,998,425; WO 96/21654; U.S. Pat. No. 5,658,903; U.S. Pat. No. 6,369,068; U.S. Pat. No. 5,739,143; U.S. Pat. No. 5,716,955; U.S. Pat. No. 6,372,741; U.S. Pat. No. 6,096,748; U.S. Pat. No. 6,414,150; U.S. Pat. No. 5,774,127; U.S. Pat. No. 6,329,526; U.S. Pat. No. 5,929,076; U.S. Pat. No. 5,756,499; U.S. Pat. No. 6,046,08; U.S. Pat. No. 6,489,325; U.S. Pat. No. 6,509,363; U.S. Pat. No. 6,610,695; U.S. Pat. No. 6,362,193; U.S. Pat. No. 6,548,520; U.S. Pat. No. 6,589,954; US 2004/097493; US 2004/209886; US 2004/09903; US 2004/09904; US 2004/54236; WO 99/61440; U.S. Pat. No. 6,649,617; U.S. Pat. No. 6,548,503; U.S. Pat. No. 6,469,018; U.S. Pat. No. 6,509,363; U.S. Pat. No. 6,809,199; U.S. Pat. No. 6,962,996; U.S. Pat. No. 6,800,626; U.S. Pat. No. 6,759,535; WO 01/38314; WO 01/38313; WO 01/38312; U.S. Pat. No. 6,759,410; WO 01/64679; WO 02/059083; WO 02/32862; WO 02/060869; WO 01/37835; WO 02/39954; WO 04/021979, now U.S. Pat. No. 6,809,199; WO 03/088972; WO 04/073628; WO 2005/123744, WO 2003/033502; WO 2003/045941; WO 2004/000846; WO 2004/014920; WO 2004/031188; WO 2004/113348; WO 2004/113347; WO 2003/087087; WO 2004/004720; WO 2004/089929; WO 2004/100946; WO 2004/078116; WO 2004/078747; WO 2004/102636; U.S. Pat. No. 6,344,476; WO 2003/068299; WO 2003/068746; WO 2003/008413; WO 2003/076405; WO 99/32463; US 2002/065296; US 2004/102636; WO 2003/064417; WO 2003/064418; WO 2003/064419; WO 2003/087096; WO 2004/004725; WO 2004/014870; WO 2004/014387; WO 2004/043367; WO 2004/02956; WO 2004/099156; WO 2002/040486; WO 2002/096426; WO 2003/002544; WO 2003/024899; WO 2003/053941; WO 2003/090912; WO 2003/091229; WO 2003/099820; WO 2004/060306; WO 2004/078756; WO 2004/013139; WO 2004/014900; WO 2003/074530; WO 2002/018379; WO 2002/064594; WO 2001/029042; WO 2003/082871; WO 2004/014907; WO 2003/020715; WO 2002/058695; WO 2003/000682; WO 2003/097062; WO 2004/101529; WO 2002/072576; WO 2004/087615; WO 2004/143119; WO 2004/077682; WO 2004/0092547; WO 2004/157877; WO 2002/072579; WO 2004/020438; WO 2004/020440; WO 2004/072072; WO 2004/058176; WO 2004/087074; WO 2003/026663; WO 2003/059891; WO 2003/068230; WO 2002/044168; WO 2002/046158; WO 2003/069248; WO 2002/042292; WO 2004/022712; WO 2004/032874; U.S. Pat. No. 6,476,031; WO 2002/100405; WO 2004/037814; WO 2004/072038; WO 2000/017204; WO 2000/017175; WO 2001/070695; WO 2002/092087; WO 2002/100405; WO 2000/26209; WO 2000/18738; WO 00/20402; WO 99/64400; WO 2000/01688; WO 2000/07980; WO 2000/07991; WO 00/06563; WO 00/12074; WO 2000/12497; WO 2000/31072; WO 2000/31063; WO 2000/23072; WO 2000/31065; WO 2000/35911; WO 2000/39116; WO 2000/43384; WO 2000/41698; WO 99/58502; WO 99/58523; WO 99/57101; WO 99/61426; WO 99/64400; WO 99/59960; WO 99/15164; WO 99/59959; WO 99/17204; WO 99/00357; WO 99/03837; WO 99/01441; WO 99/01449; WO 99/03484; WO 98/47892; WO 98/56377; WO 98/22109; WO 98/24782; WO 98/24780; WO 98/22457; WO 98/52558; WO 98/52941; WO 98/52937; WO 98/52940; WO 98/56788; WO 98/27098; WO 98/47899; WO 98/50356; WO 97/36587; WO 97/47618; WO 97/16442; WO 97/16441; WO 97/12876; WO 95/09853; WO 95/09851; WO 95/09847; WO 95/09852; WO 92/12154; WO 94/19350; Lee et al., Current Med Chem, Vol. 12, pp 2979-2994 (2005); Goldstein et al., J. Med Chem., Vol. 49, pgs 1562-1575 (2006), RO-3201195=S-[5-amino-1-(4-fluorophenyl)-1H-pyrazol-4-yl]-[3-(2,3-dihydroxypropoxy)phenyl]-methanone; Doramapimod (Birb-796/Birb796BS), WO 2004/49742; Scio-469, Scio-323; or de Dios, A. et al., J. Med. Chem., Vol. 48, pp. 2270-2273 (2005).

11. The method according to claim 7 wherein the CSBP/p38 inhibitor is selected from a compound disclosed in WO 2004/072038; WO 2005/0176964; U.S. Pat. No. 6,509,363; WO 2001/64679; and WO 2002/059083.

12. The method according to claim 7 wherein the CSBP/p38 inhibitor is RO-3201195 (S-[5-amino-1-(4-fluorophenyl)-1H-pyrazol-4-yl]-[3-(2,3-dihydroxypropoxy)phenyl]-methanone); Doramapimod (Birb-796/Birb796BS); 8-(2,6-Difluoro-phenyl)-4-(4-fluoro-2-methyl-phenyl)-2-(2-hydroxy-1-hydroxymethyl-ethylamino)-8H-pyrido[2,3-d]pyrimidin-7-one, or a pharmaceutically acceptable salt thereof; 6-(5-cyclopropylcarbamoyl-3-fluoro-2-methyl-phenyl)-N-(2,2-dimethylpropyl)-nicotinamide, or a pharmaceutically acceptable salt thereof; Scio-469, Scio-323, or VX-702.

13. A method of reducing body mass in a mammal in need thereof comprising the step of administering to said mammal an amount of a p38 kinase inhibitor effective to reduce body mass.

14. The method according to claim 13 wherein the p38 kinase inhibitor is administered with a second therapeutic agent.

15. The method according to claim 13 wherein the p38 inhibitor and/or therapeutic agent is administered orally, topically (intranasal) or via inhalation (aerosol), or both topically and via inhalation.

16. The method according to claim 13 wherein the CSBP/p38 inhibitor is selected from a compound disclosed in WO 05/073219; WO 05/073189; WO 05/073217; WO 05/07232; 2005/0038014, Angell et al.; 2004/0266839, Angell et al.; 2004/0249161, Angell et al.; 2005/0020540, Angell et al.; 2005/0176964, Aston et al.; 2005/0065195, Angell et al.; 2005/0090491, Angell et al.; 2004/0242868, Angell et al.; 2004/0267012, Angell et al.; WO 03/093248, Angell et al.; WO 03/032970, Angell et al.; WO 05/014550, Walker, A.; WO 04/010995, Angell et al.; WO 04/089875, Aston, N.; WO 04/089876, Aston, N.; WO 04/089874, Aston, et al.; U.S. Pat. No. 5,716,972; U.S. Pat. No. 5,686,455; U.S. Pat. No. 5,656,644; U.S. Pat. No. 5,916,891; U.S. Pat. No. 5,593,992; U.S. Pat. No. 6,288,062; U.S. Pat. No. 5,545,669; U.S. Pat. No. 5,559,137; U.S. Pat. No. 5,998,425; WO 96/21654; U.S. Pat. No. 5,658,903; U.S. Pat. No. 6,369,068; U.S. Pat. No. 5,739,143; U.S. Pat. No. 5,716,955; U.S. Pat. No. 6,372,741; U.S. Pat. No. 6,096,748; U.S. Pat. No. 6,414,150; U.S. Pat. No. 5,774,127; U.S. Pat. No. 6,329,526; U.S. Pat. No. 5,929,076; U.S. Pat. No. 5,756,499; U.S. Pat. No. 6,046,08; U.S. Pat. No. 6,489,325; U.S. Pat. No. 6,509,363; U.S. Pat. No. 6,610,695; U.S. Pat. No. 6,362,193; U.S. Pat. No. 6,548,520; U.S. Pat. No. 6,589,954; US 2004/097493; US 2004/209886; US 2004/09903; US 2004/09904; US 2004/54236; WO 99/61440; U.S. Pat. No. 6,649,617; U.S. Pat. No. 6,548,503; U.S. Pat. No. 6,469,018; U.S. Pat. No. 6,509,363; U.S. Pat. No. 6,809,199; U.S. Pat. No. 6,962,996; U.S. Pat. No. 6,800,626; U.S. Pat. No. 6,759,535; WO 01/38314; WO 01/38313; WO 01/38312; U.S. Pat. No. 6,759,410; WO 01/64679; WO 02/059083; WO 02/32862; WO 02/060869; WO 01/37835; WO 02/39954; WO 04/021979, U.S. Pat. No. 6,809,199; WO 03/088972; WO 04/073628; WO 2005/123744, WO 2003/033502; WO 2003/045941; WO 2004/000846; WO 2004/014920; WO 2004/031188; WO 2004/113348; WO 2004/113347; WO 2003/087087; WO 2004/004720; WO 2004/089929; WO 2004/100946; WO 2004/078116; WO 2004/078747; WO 2004/102636; U.S. Pat. No. 6,344,476; WO 2003/068299; WO 2003/068746; WO 2003/008413; WO 2003/076405; WO 99/32463; US 2002/065296; US 2004/102636; WO 2003/064417; WO 2003/064418; WO 2003/064419; WO 2003/087096; WO 2004/004725; WO 2004/014870; WO 2004/043367; WO 2004/014387; WO 2004/02956; WO 2004/099156; WO 2002/040486; WO 2002/096426; WO 2003/002544; WO 2003/024899; WO 2003/053941; WO 2003/090912; WO 2003/091229; WO 2003/099820; WO 2004/060306; WO 2004/078756; WO 2004/013139; WO 2004/014900; WO 2003/074530; WO 2002/018379; WO 2002/064594; WO 2001/029042; WO 2003/082871; WO 2004/014907; WO 2003/020715; WO 2002/058695; WO 2003/000682; WO 2003/097062; WO 2004/101529; WO 2002/072576; WO 2004/087615; WO 2004/143119; WO 2004/077682; WO 2004/0092547; WO 2004/157877; WO 2002/072579; WO 2004/020438; WO 2004/020440; WO 2004/072072; WO 2004/058176; WO 2004/087074; WO 2003/026663; WO 2003/059891; WO 2003/068230; WO 2002/044168; WO 2002/046158; WO 2003/069248; WO 2002/042292; WO 2004/022712; WO 2004/032874; U.S. Pat. No. 6,476,031; WO 2002/100405; WO 2004/037814; WO 2004/072038; WO 2000/017204; WO 2000/017175; WO 2001/070695; WO 2002/092087; WO 2002/100405; WO 2000/26209; WO 2000/18738; WO 00/20402; WO 99/64400; WO 2000/01688; WO 2000/07980; WO 2000/07991; WO 00/06563; WO 00/12074; WO 2000/12497; WO 2000/31072; WO 2000/31063; WO 2000/23072; WO 2000/31065; WO 2000/35911; WO 2000/39116; WO 2000/43384; WO 2000/41698; WO 99/58502; WO 99/58523; WO 99/57101; WO 99/61426; WO 99/64400; WO 99/59960; WO 99/15164; WO 99/59959; WO 99/17204; WO 99/00357; WO 99/03837; WO 99/01441; WO 99/01449; WO 99/03484; WO 98/47892; WO 98/56377; WO 98/22109; WO 98/24782; WO 98/24780; WO 98/22457; WO 98/52558; WO 98/52941; WO 98/52937; WO 98/52940; WO 98/56788; WO 98/27098; WO 98/47899; WO 98/50356; WO 97/36587; WO 97/47618; WO 97/16442; WO 97/16441; WO 97/12876; WO 95/09853; WO 95/09851; WO 95/09847; WO 95/09852; WO 92/12154; WO 94/19350; Lee et al., Current Med Chem, Vol. 12, pp 2979-2994 (2005); Goldstein et al., J. Med Chem., Vol. 49, pgs 1562-1575 (2006), RO-3201195=S-[5-amino-1-(4-fluorophenyl)-1H-pyrazol-4-yl]-[3-(2,3-dihydroxypropoxy)phenyl]-methanone; Doramapimod (Birb-796/Birb796BS), WO 2004/49742; Scio-469, Scio-323; or de Dios, A. et al., J. Med. Chem., Vol. 48, pp. 2270-2273 (2005).

17. The method according to claim 13 wherein the CSBP/p38 inhibitor is selected from a compound disclosed in WO 2004/072038; WO 2005/0176964; WO 2001/64679; U.S. Pat. No. 6,509,363; and WO 2002/059083.

18. The method according to claim 13 wherein the CSBP/p38 inhibitor is RO-3201195 (S-[5-amino-1-(4-fluorophenyl)-1H-pyrazol-4-yl]-[3-(2,3-dihydroxypropoxy)phenyl]-methanone); Doramapimod (Birb-796/Birb796BS); 8-(2,6-Difluoro-phenyl)-4-(4-fluoro-2-methyl-phenyl)-2-(2-hydroxy-1-hydroxymethyl-ethylamino)-8H-pyrido[2,3-d]pyrimidin-7-one, or a pharmaceutically acceptable salt thereof; 6-(5-cyclopropylcarbamoyl-3-fluoro-2-methyl-phenyl)-N-(2,2-dimethylpropyl)-nicotinamide, or a pharmaceutically acceptable salt thereof; Scio-469, Scio-323 or VX-702.

19. A method of delaying the onset of obesity, or abdominal obesity in a mammal in need of such treatment, comprising the step of administering to said mammal an amount of a p38 kinase inhibitor effective to delay the onset of obesity, or abdominal obesity.

20. A method of reducing the risk of developing obesity, or abdominal obesity in a mammal in need of such treatment, comprising the step of administering to said mammal an amount of a p38 kinase inhibitor effective to reduce the risk of developing obesity, or abdominal obesity.

21. A method of treating obesity, or abdominal obesity in a mammal in need of such treatmen comprising the step of administering to said mammal an amount of a p38 kinase inhibitor and a compound selected from the group consisting of: DP-IV inhibitors; insulin sensitizers selected from the group consisting of (i) PPAR agonists and (ii) biguanides; insulin and insulin mimetics; sulfonylureas and other insulin secretagogues; oc-glucosidase inhibitors; glucagon receptor antagonists; GLP-1, GLP-1 mimetics, and GLP-1 receptor agonists; GIP, GIP mimetics, and GIP receptor agonists; PACAP, PACAP mimetics, and PACAP receptor 3 agonists; cholesterol lowering agents selected from the group consisting of (i):G-CoA reductase inhibitors, (ii) sequestrants, (iii) nicotinyl alcohol, nicotinic acid and salts thereof, (iv) PPARot agonists, (v) PPARoc/y dual agonists, (vi) inhibitors of cholesterol absorption, (vii) acyl CoA:cholesterol acyltransferase inhibitors, and (viii) anti oxidants; PPARD agonists; said compounds being administered to the patient in an amount that is effective to treat obesity, or abdominal obesity.
